(19) United States
(12) Patent Application Publication
(10) Pub. No.: US 2013/0112218 A1
(43) Pub. Date: May 9, 2013

(54) USE OF ELLAGIC ACID AS AN ANTI-DANDRUFF AGENT

(71) Applicant: Tiphaine Derkx, Paris (FR)

(72) Inventor: Tiphaine Derkx, Paris (FR)

(21) Appl. No.: 13/582,723

(22) PCT Filed: Mar. 1, 2011
(86) PCT No.: PCT/EP11/53017

§ 371 Date: Jan. 17, 2013

Related U.S. Application Data
(60) Provisional application No. 61/313,847, filed on Mar. 15, 2010.

(30) Foreign Application Priority Data
Mar. 1, 2010 (FR) 1051443

(51) Int. Cl. A61K 8/49; A61Q 9/02

(52) U.S. Cl. 132/202; 549/278; 536/18.1; 514/453

(57) ABSTRACT

The invention relates to the use of one or more compounds selected from ellagic acid, ethers thereof, ellagic acid salts and ellagic acid ether salts, as an anti-dandruff agent; and to a cosmetic treatment method associated with such a use.

The present invention relates to the use of at least one compound selected from ellagic acid, ethers thereof, ellagic acid salts and ellagic acid ether salts, and mixtures thereof, as an anti-dandruff agent, and in particular in the cosmetic treatment of dandruff conditions related to excessive proliferation of yeasts of the Malassezia genus on the scalp. The invention also relates to a cosmetic treatment method intended for eliminating and/or reducing dandruff, in particular dandruff caused by yeasts of the Malassezia genus, which comprises the application of said compound(s) as an anti-dandruff cosmetic agent, to the scalp.

Dandruff problems affect up to 50% of the worldwide population. They affect both men and women and are perceived to have a very negative psychosocial impact. The appearance of dandruff is bothersome both from an aesthetic point of view and because of the annoyances that it causes (itching, redness, etc.), with the result that many people confronted with this problem to varying degrees wish to be free of it in an effective and permanent manner.

Dandruff corresponds to an excessive and visible desquamation of the scalp resulting from a too rapid multiplication of the epidermal cells and from abnormal maturation thereof. This phenomenon can be caused in particular by microtraumas of physical or chemical nature, such as hair treatments which are too aggressive, extreme climatic conditions, nervousness, diet, fatigue or pollution, but it has been demonstrated that dandruff conditions most commonly result from a disorder of the microflora of the scalp, and more particularly from excessive colonization by a fungus which belongs to the family of yeasts of the Malassezia genus (formerly known as Pytirosporum) and which is naturally present on the scalp.

Many anti-dandruff treatments have been developed with the principal objective of eradicating scalp Malassezia yeasts. Thus, the activity of the major anti-dandruff agents of today, such as zinc pyrithione, piroctone olamine or selenium disulphide, is based mainly on their fungicidal property.

In patent application FR 2 908 045, it is known practice to use an active ingredient rich in hydro lysable tannins (esters of polyphenolic carboxylic acids and of saccharides) for treating and/or preventing dandruff conditions by in particular combating microbial proliferation.

However, these anti-dandruff agents are not completely satisfactory in terms of effectiveness (immediate effectiveness or duration of the effect) and/or in terms of impact on the environment.

In the interests of environmental friendliness, the aim of the present application is to find other anti-dandruff agents which are not irritant for the skin and the scalp, which are more environmentally friendly, but which are just as effective compared with known anti-dandruff agents.

Prior patents describe the use of ellagic acid for its depigmenting, ultraviolet-radiation-screening, anticancer and anti-inflammatory properties.

The applicant has now found, surprisingly, that the use of at least one compound selected from ellagic acid, ethers thereof, ellagic acid salts and ellagic acid ether salts, and mixtures thereof, makes it possible to effectively treat dandruff conditions, in particular those associated with the proliferation of yeasts of the Malassezia genus, and to remedy the drawbacks of the prior art.

It has been observed that, by using one or more active agents selected from ellagic acid, ethers thereof and the salts of these compounds, it is possible to eliminate and/or reduce the number of yeasts of the Malassezia genus, the number of dandruff flakes, and the itching or redness on the scalp.

A subject of the present invention is therefore the use of one or more compounds selected from ellagic acid, ethers thereof, ellagic acid salts and ellagic acid ether salts, and mixtures thereof, as an anti-dandruff agent.

Another subject of the invention relates to a cosmetic treatment method intended for eliminating and/or reducing dandruff, in particular dandruff caused by yeasts of the Malassezia genus, which comprises the application of one or more compounds selected from ellagic acid, ethers thereof and salts of these compounds, and mixtures thereof, as an anti-dandruff agent, to the scalp.

Other features, aspects, subjects and advantages of the present invention will emerge even more clearly on reading the description and the examples which follow.

The anti-dandruff agent used according to the invention is selected from ellagic acid, ethers thereof and salts of these compounds.

Ellagic acid, also known as: 2,3,7,8-tetrahydroxy(1)benzopyrano(5,4,3-cde)(1)benzopyran-5,10-dione, is a well-known molecule present in the plant kingdom. Reference may be made to the publication of the Merck Index 20^{th} edition (1996), No. 3588.

Ellagic acid has the following chemical formula:

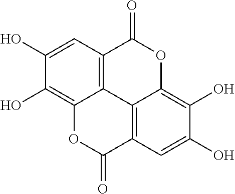 [SMILES: O=c1oc2c(O)c(O)cc3c(=O)oc4c(O)c(O)cc1c4c23]

which comprises four fused rings.

Ellagic acid is commercially available, in particular from the company Sigma, France.

A method for purifying ellagic acid and also the purified ellagic acids obtained by means of such a method are known from document FR-A-1 478 523.

The ellagic acid ether(s) that can be used according to the invention is (are) preferably selected from the mono-, di-, tri- or polyethers obtained by etherification of one or more hydroxyl groups (one of the four OH groups of ellagic acid) of ellagic acid to give one or more OR groups, R being selected from C₂-C₂₀ alkyl groups, polyoxyalkylene groups, and in particular polyoxyethylene and/or polyoxypropylene groups, and groups derived from one or more monosaccharides or polysaccharides, such as, for example, the group having the following formula:

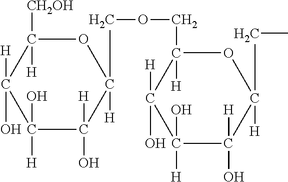

In the case of the di-, tri- or polyethers of ellagic acid, the R groups as defined above may be identical or different.

Such ethers are described in U.S. Pat. No. 5,073,545. Preferably, these ellagic acid ethers are selected from 3,4-di-O-methyl ellagic acid, 3,3′,4-tri-O-methyl ellagic acid and 3,3′-di-O-methyl ellagic acid.

The ellagic acid salt(s) and/or the ellagic acid ether salt(s) that can be used according to the invention are preferably selected from the alkali or alkaline-earth metal salts, such as the sodium, potassium, calcium and magnesium salt, the ammonium salt and the amine salts, such as the salts of triethanolamine, of monoethanolamine, of arginine and of lysine. Preferably, the ellagic acid salt(s) and/or the ellagic acid ether salt(s) that can be used according to the invention are selected from the alkali or alkaline-earth metal salts, in particular the sodium, potassium, calcium and magnesium salts.

Among all the compounds mentioned, ellagic acid or a salt thereof is preferably used as anti-dandruff agent.

The abovementioned compound(s) used according to the invention as anti-dandruff agents is (are) generally used by topical application.

In particular, this or these compound or compounds can be used as anti-dandruff agents in a cosmetic composition that can be in any of the galenical forms normally used for topical application.

Preferably, the compound(s) selected from ellagic acid, ethers thereof, ellagic acid salts and ellagic acid ether salts, and mixtures thereof, represent from 0.01% to 10%, preferably from 0.1% to 5%, and even better still from 0.2% to 2% by weight, relative to the total weight of the cosmetic composition.

The cosmetic composition used according to the invention may be a rinse-out or leave-in composition. In particular, it may be a shampoo, a cream, a mousse (aerosol or nonaerosol), a paste, a gel, an emulsion, a lotion, a stick, etc. Preferably, the cosmetic composition is a shampoo or a gel.

The cosmetic composition according to the invention may comprise one or more thickeners and/or one or more surfactants selected from anionic, non-ionic and/or amphoteric or zwitterionic surfactants. These thickeners and surfactants are defined hereinafter.

In a first preferred embodiment, the anti-dandruff compound(s) used according to the invention and as defined above, are used in a gelled cosmetic composition (or gel). In this case, the cosmetic composition comprises one or more compounds selected from ellagic acid, ethers thereof, ellagic acid salts and ellagic acid ether salts, and mixtures thereof, and one or more thickeners.

For the purpose of the present invention, the term “thickener” means an agent which, introduced at 1% by weight into an aqueous solution or aqueous-alcoholic solution containing 30% of ethanol, at pH=7, makes it possible to achieve a viscosity of at least 100 cPs, preferably of at least 500 cPs, at 25° C., and a shear rate of 1 s⁻¹. This viscosity can be measured using a cone/plate viscometer (Haake R600 rheometer or the like).

The thickener(s) is (are) preferably present in a sufficient amount to obtain a gel.

The term “gel” or “gelled composition” means a composition having a viscosity ranging from 100 cPs to 500 000 cPs, better still from 200 cPs to 100 000 cPs, at ambient temperature (25° C.), at atmospheric pressure (1 bar) and at a shear rate of 1 s⁻¹. This viscosity can be measured using a cone/plate viscometer (Haake R600 rheometer or the like).

This particular form allows the composition to remain well localized at the point of application. Thus, the composition does not run, thereby reducing the risks of the composition coming into contact with the eyes.

Preferably, the cosmetic composition comprises from 0.1% to 20% by weight, and even better still from 0.2% to 10% by weight of thickener(s), relative to the total weight of the composition.

The thickener(s) can be selected from sodium chloride, fatty acid amides obtained from a C₁₀-C₃₀ carboxylic acid (coconut acid monoisopropanolamide, diethanolamide or monoethanolamide, oxyethylenated carboxylic acid monoethanolamide alkyl ether), non-ionic cellulosic thickeners (hydroxyethylcellulose, hydroxypropylcellulose, carboxymethylcellulose), guar gum and non-ionic derivatives thereof (hydroxypropylguar), gums of microbial origin (xanthan gum, scleroglucan gum), crosslinked or non-crosslinked homopolymers and copolymers based on acrylic acid, on methacrylic acid or on acrylamidopropanesulphonic acid, and the associative polymers as described below.

The associative polymer(s) that can be used according to the invention are water-soluble polymers capable, in an aqueous medium, of reversibly associating with one another or with other molecules.

Their chemical structure comprises hydrophilic zones and hydrophobic zones, characterized by at least one fatty chain preferably containing from 10 to 30 carbon atoms.

The associative polymer(s) that can be used according to the invention may be of anionic, cationic, amphoteric or non-ionic type, such as the polymers sold under the names Pemulen TR1 or TR2 by the company Goodrich (INCI: Acrylates/C₁₀₋₃₀ Alkyl Acrylate Crosspolymer), Salcare SC90 by the company Ciba, Aculyn 22, 28, 33, 44 or 46 by the company Rohm & Haas, and Elfacos T210 and T212 by the company Akzo.

Among all the thickeners mentioned, use is preferably made of acrylic acid-based or methacrylic acid-based homopolymers and copolymers, which are preferably crosslinked, and/or fatty acid amides obtained from a C₁₀-C₃₀ carboxylic acid.

In a second preferred embodiment, the anti-dandruff compound(s) used according to the invention, and as defined above, is (are) used in a shampoo composition. In this case, the composition comprises one or more compounds selected from ellagic acid, ethers thereof, ellagic acid salts and ellagic acid ether salts, and mixtures thereof, and one or more surfactants selected from anionic, non-ionic, and amphoteric or zwitterionic surfactants, and mixtures thereof.

These surfactants are in particular mentioned in the CTFA (edition 2004) under the name “surfactant—cleansing agent”.

The term “anionic surfactant” means a surfactant comprising, as ionic or ionisable groups, only anionic groups. These anionic groups are preferably selected from CO₂H, CO₂⁻, SO₃H, SO₃⁻, OSO₃H, OSO₃⁻, O₂PO₂H, O₂PO₂H and O₂PO₂²⁻ groups.

The anionic surfactant(s) that may be used in the compositions of the invention is (are) in particular selected from alkyl sulphates, alkyl ether sulphates, alkylamido ether sulphates, alkylaryl polyether sulphates, monoglyceride sulphates, alkyl sulphonates, alkylamide sulphonates, alkylarylsulphonates, alpha-olefin sulphonates, paraffin sulphonates, alkylsulphosuccinates, alkyl ether sulphosuccinates, alkylamide sulphosuccinates, alkyl sulphoacetates, acylsarcosinates, acylglutamates, alkyl sulphosuccinamates, acyl isethionates and N-acyltaurates; salts of alkyl monoesters of polyglycoside-polycarboxylic acids, acyllactylates, salts of D-galactosiduronic acids, salts of alkyl ether carboxylic acids, salts of alkyl aryl ether carboxylic acids, salts of alkylamido ether carboxylic acids; and the corresponding non-salified forms of all these compounds; the alkyl and acyl groups of all these compounds containing from 6 to 24 carbon atoms and the aryl group denoting a phenyl group.

Some of these anionic surfactants may be oxyethylenated and then preferably comprise from 1 to 50 ethylene oxide units.

The salts of C₆₋₂₄ alkyl monoesters of polyglycoside-polycarboxylic acids can be selected from C₆₋₂₄ alkyl polyglycoside-citrates, C₆₋₂₄ alkyl polyglycoside-tartrates and C₆₋₂₄ alkyl polyglycoside-sulphosuccinates.

When the anionic surfactant(s) (iii) are in salt form, it (they) can be selected from alkali metal salts, such as the sodium or potassium salt, and preferably the sodium salt, ammonium salts, amine salts, and in particular aminoalcohol salts, and alkaline-earth metal salts such as the magnesium salt.

By way of example of aminoalcohol salts, mention may in particular be made of monoethanolamine, diethanolamine and triethanolamine salts, monoisopropanolamine, diisopropanolamine or triisopropanolamine salts, and 2-amino-2-methyl-1-propanol, 2-amino-2-methyl-1,3-propanediol and tris(hydroxymethyl)aminomethane salts.

Alkali metal or alkaline-earth metal salts, and in particular sodium or magnesium salts, are preferably used.

(C₆₋₂₄)alkyl sulphates, (C₆₋₂₄)alkyl ether sulphates, which are optionally oxyethylenated, and mixtures thereof, in particular in the form of alkali metal or alkaline-earth metal salts, ammonium salts and aminoalcohol salts, as described above, are preferably used. More preferably, the anionic surfactant(s) is (are) selected from (C₁₀-C₂₀)alkyl ether sulphates, and in particular sodium lauryl ether sulphate comprising 2.2 mol of ethylene oxide.

When they are present, the amount of the anionic surfactant(s) preferably ranges from 0.1% to 50% by weight, even better still from 4% to 30% by weight, relative to the total weight of the composition.

Examples of non-ionic surfactants that can be used in the cosmetic composition used according to the invention are described, for example, in “Handbook of Surfactants” by M. R. Porter, published by Blackie & Son (Glasgow and London), 1991, pp. 116-178. They are selected in particular from alcohols, alpha-diols, (C₁₋₂₀)alkylphenols or polyethoxylated, polypropoxylated and/or polyglycerolated fatty acids; having a fatty chain containing, for example, from 8 to 18 carbon atoms, it being possible for the number of ethylene oxide and/or propylene oxide groups to range in particular from 2 to 50 and it being possible for the number of glycerol groups to range in particular from 2 to 30.

Mention may also be made of copolymers of ethylene oxide and of propylene oxide, optionally oxyethylenated fatty acid esters of sorbitan, fatty acid esters of sucrose, polyoxyalkylenated fatty acid esters, optionally oxyalkylenated alkylpolyglycosides, alkylglucoside esters, N-alkylglucamine and N-acylmethylglucamine derivatives, aldobionamides and amine oxides.

Unless otherwise mentioned, the term “fatty” compound (for example a fatty acid) denotes a compound comprising, in its main chain, at least one saturated or unsaturated alkyl chain containing at least 8 carbon atoms, preferably from 8 to 30 carbon atoms, and even better still from 10 to 22 carbon atoms.

When they are present, the amount of the non-ionic surfactant(s) preferably ranges from 0.01% to 20% by weight, even better still from 0.2% to 10% by weight, relative to the total weight of the cosmetic composition.

The amphoteric or zwitterionic surfactant(s) that can be used in the present invention can in particular be derivatives of secondary or tertiary aliphatic amines, which are optionally quaternized, in which the aliphatic group is a linear or branched chain containing from 8 to 22 carbon atoms, said amine derivatives containing at least one anionic group such as, for example, a carboxylate, sulphonate, sulphate, phosphate or phosphonate group. Mention may in particular be made of (C₈-C₂₀)alkylbetaines, sulphobetaines, (C₈-C₂₀ alkyl)amido(C₂-C₈ alkyl)betaines or (C₈-C₂₀ alkyl)amido(C₂-C₈ alkyl)sulphobetaines.

Among the optionally quaternized secondary or tertiary aliphatic amine derivatives that can be used, as defined above, mention may also be made of the compounds of respective structures (A1) and (A2):

Rₐ—CONHCH₂CH₂—N⁺(R_{b})(R_{c})(CH₂COO⁻) (A1)

in which:

Rₐ represents a C₁₀-C₃₀ alkyl or alkenyl group derived from an acid Rₐ—COOH preferably present in hydrolysed coconut oil, a heptyl group, a nonyl group or an undecyl group,

R_{b} represents a beta-hydroxyethyl group, and

R_{c} represents a carboxymethyl group;

and

Rₐ′—CONHCH₂CH₂—N(B)(B′) (A2)

in which:

B represents —CH₂CH₂OX′,

B′ represents —(CH₂)_{z}—Y′, with z=1 or 2,

X′ represents the —CH₂—COOH, CH₂—COOZ′, —CH₂CH₂—COOH or —CH₂CH₂—COOZ′ group, or a hydrogen atom,

Y′ represents —COOH, —COOZ′, or the —CH₂—CHOH—SO₃H or —CH₂—CHOH—SO₃Z′ group,

Z′ represents an ion derived from an alkali or alkaline-earth metal, such as sodium, potassium or magnesium; an ammonium ion; or an ion derived from an organic amine and in particular from an aminoalcohol, such as mono-, di- and triethanolamine, mono-, di- or triisopropanolamine, 2-amino-2-methyl-1-propanol, 2-amino-2-methyl-1,3-propanediol and tris(hydroxymethyl)aminomethane, Rₐ′ represents a C₁₀-C₃₀ alkyl or alkenyl group of an acid Rₐ′COOH preferably present in hydrolysed linseed oil or coconut oil, an alkyl group, which is in particular C₁₇, and its iso form, or an unsaturated C₁₇ group.

These compounds are classified in the CTFA dictionary, 5th edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid and cocoamphodipropionic acid.

By way of example, mention may be made of the cocoamphodiacetate sold by the company Rhodia under the trade name Miranol® C2M concentrate.

Among the amphoteric or zwitterionic surfactants mentioned above, (C₈-C₂₀ alkyl)betaines, (C₈-C₂₀ alkyl)amido(C₂-C₈ alkyl)betaines such as cocoylamidopropylbetaine, and mixtures thereof, are preferably used. More preferably, the amphoteric or zwitterionic surfactant(s) is (are) selected from cocoylamidopropylbetaine and cocoylbetaine.

When they are present, the amount of the amphoteric or zwitterionic surfactant(s) is preferably included in the range of from 0.01% to 20% by weight, even better still from 0.5% to 10% by weight, relative to the total weight of the cosmetic composition.

Preferably, the total amount of anionic, non-ionic, and/or amphoteric or zwitterionic surfactants is at least 3% by weight, relative to the total weight of the composition. Even more preferably, the composition according to the invention has a total content of anionic, non-ionic, and/or amphoteric or zwitterionic surfactants ranging from 4% to 50% by weight, even better still from 4% to 20% by weight, relative to the total weight of the composition.

In one preferred embodiment, the cosmetic composition used according to the invention is a shampoo as defined above comprising one or more thickeners. The thickener(s) is (are) preferably selected from those defined above. The thickener(s) can also be present in concentrations as described above.

The cosmetic composition used according to the invention generally comprises a cosmetically acceptable medium. The medium consists of water and optionally of one or more cosmetically acceptable organic solvents.

The organic solvent(s) can be selected from C₁-C₄ lower alcohols, such as ethanol, isopropanol, tert-butanol or n-butanol; polyols such as glycerol, propylene glycol, hexylene glycol (or 2-methyl-2,4-pentanediol) and polyethylene glycols; polyol ethers, for instance dipropylene glycol monomethyl ether; and mixtures thereof.

Preferably, the cosmetic composition used according to the invention contains an amount of organic solvents ranging from 0.05% to 60%, preferably from 0.5% to 50%, and even better still from 1% to 40% by weight, relative to the total weight of the cosmetic composition.

The cosmetic composition used according to the invention may also comprise one or more conditioning agents.

The term “conditioning agent” denotes, according to the present invention, any composition which makes it possible to improve the cosmetic properties of the hair, in particular the softness, the disentangling, the feel or the static electricity.

Preferably, the conditioning agent is selected from the group comprising cationic polymers, cationic surfactants, silicones, such as organosiloxanes, linear or branched C₈-C₃₀ hydrocarbons, linear or branched C₈-C₃₀ fatty alcohols, esters of C₈-C₃₀ fatty acid and of C₁-C₃₀ alcohol, and in particular esters of C₈-C₃₀ fatty acid and of C₈-C₃₀ fatty alcohol, esters of C₁-C₇ acid or diacid and of C₈-C₃₀ fatty alcohol, ceramides or ceramide analogues, and mixtures of these compounds.

The term “cationic polymer” means a polymer which is positively charged when it is contained in the cosmetic composition according to the invention. This polymer can carry one or more permanent positive charges or contain one or more functions that are cationizable in the cosmetic composition according to the invention.

The cationic polymer(s) that can be used as conditioning agents according to the present invention is (are) preferably selected from polymers comprising primary, secondary, tertiary and/or quaternary amine groups which are part of the polymer chain or directly connected thereto, and having a molecular weight of between 500 and approximately 5 000 000, and preferably between 1000 and 3 000 000.

When the conditioning agent is a cationic polymer, it is preferably selected from those which contain units comprising primary, secondary, tertiary and/or quaternary amine groups that can either be part of the main polymer chain or be carried by a side substituent directly connected to said chain.

Among the cationic polymers, mention may more particularly be made of polymers of the polyamine, polyamino amide and polyquaternary ammonium type. These are known products. They are, for example, described in French patents Nos. 2 505 348 and 2 542 997.

Among these polymers, mention may be made of:

(1) Homopolymers or copolymers derived from acrylic or methacrylic esters or amides and comprising at least one of the units of the following formulae:

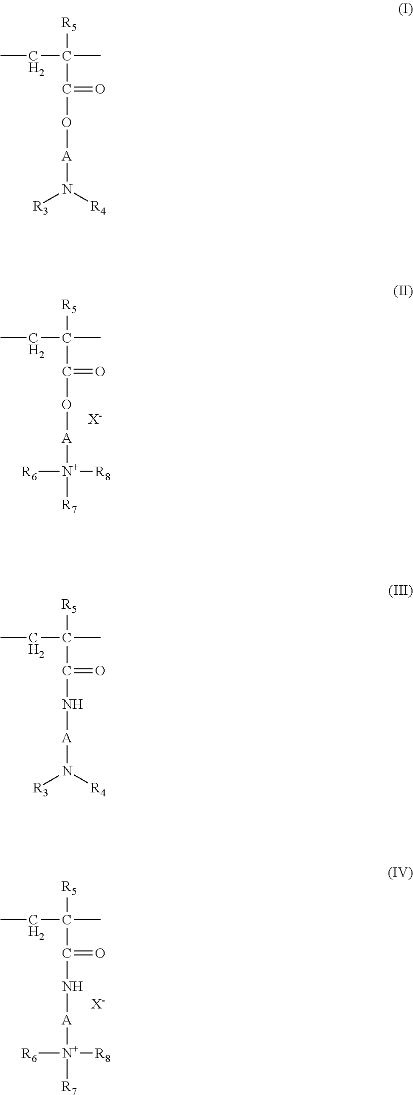

in which:

R₃ and R₄, which may be identical or different, represent a hydrogen atom or an alkyl group containing from 1 to 6 carbon atoms, and preferably methyl or ethyl;

R₅, which may be identical or different, denote a hydrogen atom or a CH₃ group;

A, which may be identical or different, represent a linear or branched alkyl group containing from 1 to 6 carbon atoms, preferably 2 or 3 carbon atoms, or a hydroxyalkyl group containing from 1 to 4 carbon atoms;

R₆, R₇ and R₈, which may be identical or different, represent an alkyl group containing from 1 to 18 carbon atoms or a benzyl group, and preferably an alkyl group containing from 1 to 6 carbon atoms;

X⁻ denotes an anion derived from a mineral or organic acid, such as a methosulphate anion or a halide such as chloride or bromide.

The copolymers of family (1) can also contain one or more units derived from comonomers which may be selected from the family of acrylamides, methacrylamides, diacetone acrylamides, acrylamides and methacrylamides substituted on the nitrogen with lower (C₁-C₄) alkyls, acrylic or methacrylic acids or esters thereof, vinyllactams such as vinylpyrrolidone or vinylcaprolactam, and vinyl esters.

Thus, among these copolymers of family (1), mention may be made of:
copolymers of acrylamide and of dimethylaminoethyl methacrylate quaternized with dimethyl sulphate or with a dimethyl halide, such as the product sold under the name Hercofloc by the company Hercules,
the copolymers of acrylamide and of methacryloyloxy-ethyltrimethylammonium chloride described, for example, in patent application EP-A-080 976 and sold under the name Bina Quat P 100 by the company Ciba Geigy,
the copolymer of acrylamide and of methacryloyloxy-ethyltrimethylammonium methosulphate sold under the name Reten by the company Hercules,
quaternized or non-quaternized vinylpyrrolidone/dialkylaminoalkyl acrylate or methacrylate copolymers, such as the products sold under the name Gafquat by the company ISP, such as, for example, Gafquat 734 or Gafquat 755, or alternatively the products known as Copolymer 845, 958 and 937. These polymers are described in detail in French patents 2 077 143 and 2 393 573,
dimethylaminoethyl methacrylate/vinylcaprolactam/vinylpyrrolidone terpolymers, such as the product sold under the name Gaffix VC 713 by the company ISP,
vinylpyrrolidone/methacrylamidopropyldimethylamine copolymers sold in particular under the name Styleze CC 10 by the company ISP,
quaternized vinylpyrrolidone/dimethylaminopropyl-methacrylamide copolymers, such as the product sold under the name Gafquat HS 100 by the company ISP, and
crosslinked polymers of methacryloyloxy(C₁-C₄)alkyl(C₁-C₄)trialkylammonium salts, such as the polymers obtained by homopolymerization of dimethylaminoethyl methacrylate quaternized with methyl chloride, or by copolymerization of acrylamide with dimethylaminoethyl methacrylate quaternized with methyl chloride, the homopolymerization or copolymerization being followed by crosslinking with a compound comprising an olefinic unsaturation, in particular methylenebisacrylamide. Use may more particularly be made of an acrylamide/methacryloyloxyethyltrimethylammonium chloride crosslinked copolymer (20/80 by weight) in the form of a dispersion containing 50% by weight of said copolymer in mineral oil. This dispersion is marketed under the name Salcare® SC 92 by the company Ciba. Use may also be made of a crosslinked homopolymer of methacryloyloxyethyltrimethylammonium chloride, for example as a dispersion in mineral oil or in a liquid ester. These dispersions are marketed under the names Salcare® SC 95 and Salcare® SC 96 by the company Ciba.

(2) Polymers constituted of piperazinyl units and of divalent alkylene or hydroxyalkylene groups containing straight or branched chains, optionally interrupted with oxygen, sulphur or nitrogen atoms or with aromatic or heterocyclic rings, and also the oxidation and/or quaternization products of these polymers. Such polymers are described, in particular, in French patents 2 162 025 and 2 280 361.

(3) Water-soluble polyamino amides prepared in particular by polycondensation of an acidic compound with a polyamine; these polyamino amides can be crosslinked with an epihalohydrin, a diepoxide, a dianhydride, an unsaturated dianhydride, a bis-unsaturated derivative, a bis-halohydrin, a bis-azetidinium, a bis-haloacyldiamine, a bis-alkyl halide or alternatively with an oligomer resulting from the reaction of a difunctional compound which is reactive with a bis-halohydrin, a bis-azetidinium, a bis-haloacyldiamine, a bis-alkyl halide, an epihalohydrin, a diepoxide or a bis-unsaturated derivative; the crosslinking agent being used in proportions ranging from 0.025 to 0.35 mol per amine group of the polyamino amide; these polyamino amides can be alkylated or, if they contain one or more tertiary amine functions, they can be quaternized. Such polymers are described, in particular, in French patents 2 252 840 and 2 368 508.

(4) The polyamino amide derivatives resulting from the condensation of polyalkylene polyamines with polycarboxylic acids, followed by alkylation with bifunctional agents. Mention may be made, for example, of adipic acid/dialkylaminohydroxyalkyl-dialkylenetriamine polymers in which the alkyl groups contain from 1 to 4 carbon atoms and preferably denote a methyl, ethyl or propyl group, and the alkylene groups contain from 1 to 4 carbon atoms and preferably denote the ethylene group. Such polymers are in particular described in French patent 1 583 363.

Among these derivatives, mention may be made more particularly of the adipic acid/dimethylaminohydroxypropyldiethylenetriamine polymers sold under the name Cartaretine F, F4 or F8 by the company Sandoz.

(5) The polymers obtained by reaction of a polyalkylene polyamine containing two primary amine groups and at least one secondary amine group with a dicarboxylic acid selected from diglycolic acid and saturated aliphatic dicarboxylic acids having from 3 to 8 carbon atoms. The molar ratio between the polyalkylene polyamine and the dicarboxylic acid is between 0.8:1 and 1.4:1; the polyamino amide resulting therefrom being reacted with epichlorohydrin in a molar ratio of epichlorohydrin relative to the secondary amine group of the polyamino amide of between 0.5:1 and 1.8:1. Such polymers are described in particular in U.S. Pat. Nos. 3,227,615 and 2,961,347.

Polymers of this type are sold in particular under the name

Hercosett 57 by the company Hercules Inc. or alternatively under the name PD 170 or Delsette 101 by the company Hercules in the case of the adipic acid/epoxypropyl/diethylenetriamine copolymer.

(6) Cyclopolymers of alkyldiallylamine or of dialkyldiallylammonium, such as homopolymers or copolymers containing, as main constituent of the chain, units corresponding to formula (V) or (VI):

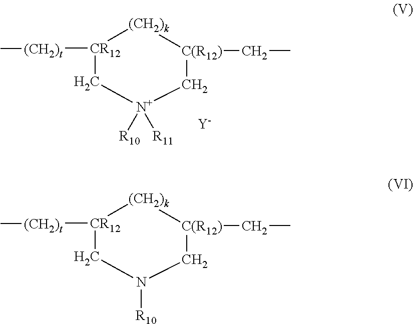

in which formulae: k and t are equal to 0 or 1, the sum k+t being equal to 1; R₁₂ denotes a hydrogen atom or a methyl group; R₁₀ and R₁₁, independently of each other, denote an alkyl group having from 1 to 6 carbon atoms, a hydroxyalkyl group in which the alkyl group preferably has 1 to 5 carbon atoms, a lower (C₁-C₄) amidoalkyl group, or R₁₀ and R₁₁ can denote, together with the nitrogen atom to which they are attached, heterocyclic groups such as piperidinyl or morpholinyl; Y⁻ is an anion such as bromide, chloride, acetate, borate, citrate, tartrate, bisulphate, bisulphite, sulphate or phosphate. These polymers are described in particular in French patent 2 080 759 and in its Certificate of Addition 2 190 406.

R₁₀ and R₁₁, independently of each other, preferably denote an alkyl group containing from 1 to 4 carbon atoms.

Among the polymers defined above, mention may be made more particularly of the dimethyldiallylammonium chloride homopolymer sold under the name Merquat 100 by the company Nalco (and its homologues of low weight-average molecular mass) and the copolymers of diallyldimethylammonium chloride and of acrylamide, sold under the name Merquat 550.

(7) The quaternary diammonium polymer containing repeating units corresponding to formula (VII):

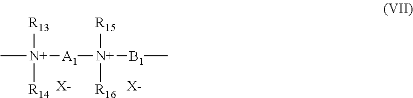

in which formula (VII):

R₁₃, R₁₄, R₁₅ and R₁₆, which may be identical or different, represent aliphatic, alicyclic or arylaliphatic groups containing from 1 to 20 carbon atoms or lower (C₁-C₄) hydroxyalkylaliphatic groups, or alternatively R₁₃, R₁₄, R₁₅ and R₁₆, together or separately, constitute, with the nitrogen atoms to which they are attached, heterocycles optionally containing a second heteroatom other than nitrogen, or alternatively R₁₃, R₁₄, R₁₅ and R₁₆ represent a linear or branched C₁-C₆ alkyl group substituted with a nitrile, ester, acyl or amide group or a group —CO—O—R₁₇-D or —CO—NH—R₁₇-D where R₁₇ is an alkylene containing from 1 to 10 carbon atoms and D is a quaternary ammonium group;

A₁ and B₁ represent polymethylene groups containing from 2 to 20 carbon atoms which may be linear or branched, saturated or unsaturated, and which may contain, linked to or intercalated in the main chain, one or more aromatic rings or one or more oxygen or sulphur atoms or sulphoxide, sulphone, disulphide, amino, alkylamino, hydroxyl, quaternary ammonium, ureido, amide or ester groups, and

X⁻ denotes an anion derived from a mineral or organic acid;

A₁, R₁₃ and R₁₅ can form, with the two nitrogen atoms to which they are attached, a piperazine ring; in addition, if A₁ denotes a linear or branched, saturated or unsaturated alkylene or hydroxyalkylene group, B₁ can also denote a group

—(CH₂)ₙ—CO-D-OC—(CH₂)ₚ—

in which:

n and p are integers ranging from 2 to 20 approximately,

D denotes:

a) a glycol residue of formula: —O—Z—O—, where Z denotes a linear or branched hydrocarbon-based group or a group corresponding to one of the following formulae:

—(CH₂—CH₂—O)_{X}—CH₂—CH₂—

—[CH₂—CH(CH₃)—O]_{y}—CH₂—CH(CH₃)—

where x and y denote an integer from 1 to 4, representing a defined and unique degree of polymerization or any number from 1 to 4 representing an average degree of polymerization;

b) a bis-secondary diamine residue such as a piperazine derivative;

c) a bis-primary diamine residue of formula: —NH—Y—NH—,

where Y denotes a linear or branched hydrocarbon-based group, or alternatively the divalent group

—CH₂—CH₂—S—S—CH₂—CH₂—;

d) a ureylene group of formula: —NH—CO—NH—.

Preferably, X⁻ is an anion such as chloride or bromide.

These polymers generally have a number-average molecular mass of between 1000 and 100 000.

Polymers of this type are described in particular in French patents 2 320 330, 2 270 846, 2 316 271, 2 336 434 and 2 413 907 and U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945 and 4,027,020.

Use may more particularly be made of polymers which are constituted of repeating units corresponding to formula (VIII):

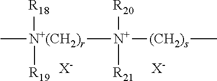

in which: R₁₈, R₁₉, R₂₀ and R₂₁, which may be identical or different, denote an alkyl or hydroxyalkyl group containing from 1 to 4 carbon atoms approximately, r and s are integers ranging from 2 to 20 approximately, and X⁻ is an anion derived from a mineral or organic acid.

A compound of formula (VIII) which is particularly preferred is the compound for which R₁₈, R₁₉, R₂₀ and R₂₁ represent a methyl group and r=3, s=6 and X═Cl, referred to as hexadimethrine chloride according to the INCI nomenclature (CTFA).

(8) Polyquaternary ammonium polymers constituted of units of formula (IX):

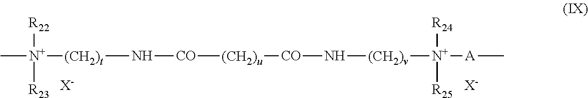

in which formula:

R₂₂, R₂₃, R₂₄ and R₂₅, which may be identical or different, represent a hydrogen atom or a methyl, ethyl, propyl, β-hydroxyethyl, β-hydroxypropyl or —CH₂CH₂(OCH₂CH₂)ₚOH radical, where p is equal to 0 or to an integer ranging from 1 to 6, with the proviso that R₂₂, R₂₃, R₂₄ and R₂₅ do not simultaneously represent a hydrogen atom,

t and u, which may be identical or different, are integers ranging from 1 to 6,

v is equal to 0 or to an integer ranging from 1 to 34,

X⁻ denotes an anion such as a halide,

A denotes a group of a dihalide or preferably represents —CH₂—CH₂—O—CH₂—CH₂—.

Such compounds are in particular described in patent application EP-A-122 324.

Among these products, mention may, for example, be made of the products Mirapol® A 15, Mirapol® AD1, Mirapol® AZ1 and Mirapol® 175 sold by the company Miranol.

(9) Quaternary polymers of vinylpyrrolidone and of vinylimidazole, such as, for example, the products sold under the names Luviquat® FC 905, FC 550 and FC 370 by the company BASF.

(10) Cationic polysaccharides, in particular cationic celluloses and cationic cellulose derivatives and cationic galactomannan gums.

Among the cationic polysaccharides, mention may more particularly be made of cellulose ether derivatives comprising quaternary ammonium groups, cationic cellulose copolymers or cellulose derivatives grafted with a water-soluble quaternary ammonium monomer and cationic galactomannan gums.

The cellulose ether derivatives comprising quaternary ammonium groups are described in French Patent 1 492 597. These polymers are also defined in the CTFA dictionary as hydroxyethylcellulose quaternary ammoniums that have reacted with an epoxide substituted with a trimethylammonium group.

The cationic cellulose copolymers or the cellulose derivatives grafted with a water-soluble quaternary ammonium monomer are described especially in U.S. Pat. No. 4,131,576, such as hydroxyalkylcelluloses, for instance hydroxymethyl-, hydroxyethyl- or hydroxypropylcelluloses grafted especially with a methacryloylethyltrimethylammonium, methacrylamido-propyltrimethylammonium or dimethyldiallylammonium salt.

The cationic galactomannan gums are described more particularly in U.S. Pat. Nos. 3,589,578 and 4,031,307, in particular guar gums containing trialkylammonium cationic groups. Use is made, for example, of guar gums modified with a salt (e.g. chloride) of 2,3-epoxypropyltrimethylammonium.

Other cationic polymers that can be used in the context of the invention are cationic proteins or cationic protein hydrolysates, polyalkyleneimines, in particular polyethyleneimines, polymers containing vinylpyridine or vinylpyridinium units, condensates of polyamines and of epichlorohydrin, quaternary polyureylenes and chitin derivatives.

The cationic proteins or protein hydrolysates are in particular chemically modified polypeptides bearing quaternary ammonium groups at the end of the chain or grafted onto said chain. Their molecular mass may range, for example, from 1500 to 10 000, and in particular from 2000 to 5000 approximately. Among these compounds, mention may in particular be made of:
hydrolysates of collagen bearing triethylammonium groups, such as the products sold under the name Quat-Pro E by the company Maybrook and called, in the CTFA dictionary, Triethonium Hydrolyzed Collagen Ethosulphate;
hydrolysates of collagen bearing trimethylammonium and trimethylstearylammonium chloride groups, sold under the name Quat-Pro S by the company Maybrook and called, in the CTFA dictionary, Steartrimonium Hydrolyzed Collagen;
hydrolysates of animal proteins bearing trimethylbenzylammonium groups, such as the products sold under the name Crotein BTA by the company Croda and called, in the CTFA dictionary, Benzyltrimonium hydrolyzed animal protein;
hydrolysates of proteins bearing, on the polypeptide chain, quaternary ammonium groups comprising at least one alkyl group containing from 1 to 18 carbon atoms.

Among these protein hydrolysates, mention may be made, inter alia, of:
Croquat L, the quaternary ammonium groups of which comprise a C₁₂ alkyl group;
Croquat M, the quaternary ammonium groups of which comprise C₁₀-C₁₈ alkyl groups;
Croquat S, the quaternary ammonium groups of which comprise a C₁₋₈ alkyl group;
Crotein Q, the quaternary ammonium groups of which comprise at least one alkyl group containing from 1 to 18 carbon atoms.

These various products are sold by the company Croda.

Other quaternized proteins or hydrolysates are, for example, those corresponding to formula (X):

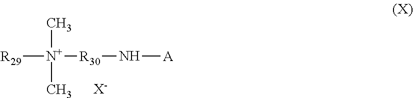

in which X⁻ is an anion of an organic or mineral acid, A denotes a protein residue derived from collagen protein hydrolysates, R₂₉ denotes a lipophilic group containing up to 30 carbon atoms, R₃₀ represents an alkylene group containing from 1 to 6 carbon atoms. Mention may, for example, be made of the products sold by the company Inolex, under the name Lexein QX 3000, called, in the CTFA dictionary, Cocotrimonium Collagen Hydrolysate.

Mention may also be made of quaternized plant proteins, such as wheat, corn or soya proteins: as quaternized wheat proteins, mention may be made of those sold by the company Croda under the names Hydrotriticum WQ or QM, called, in the CTFA dictionary, Cocodimonium Hydrolysed wheat protein; Hydrotriticum QL, called, in the CTFA dictionary, Laurdimonium hydrolysed wheat protein; or else Hydrotriticum QS, called, in the CTFA dictionary, Steardimonium hydrolysed wheat protein.

Among all the cationic polymers that may be used in the context of the present invention, it is preferred to use cationic cyclopolymers as defined above, in particular the dimethyldiallylammonium chloride homopolymers or copolymers sold under the names Merquat 100, Merquat 550 and Merquat S by the company Nalco, and quaternary vinylpyrrolidone and vinylimidazole polymers, cationic polysaccharides and mixtures thereof.

The conditioning agent(s) that can be used according to the invention can be selected from cationic surfactants.

The term “cationic surfactant” means a surfactant which is positively charged when it is contained in the composition according to the invention. This surfactant can carry one or more permanent positive charges or contain one or more functions that are cationizable in the composition according to the invention.

The cationic surfactant(s) that can be used as conditioning agents according to the present invention are preferably selected from optionally polyoxyalkylenated primary, secondary or tertiary fatty amines, or salts thereof, quaternary ammonium salts, and mixtures thereof.

Fatty amines generally comprise at least one C₈-C₃₀ hydrocarbon-based chain. Among the fatty amines that can be used according to the invention, mention may, for example, be made of stearylamidopropyldimethylamine and distearylamine.

By way of quaternary ammonium salts, mention may in particular be made, for example, of:
those corresponding to general formula (XI) below:

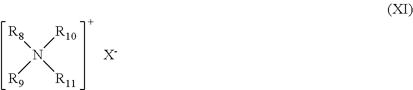

in which the groups R₈ to R₁₁, which may be identical or different, represent a linear or branched aliphatic group containing from 1 to 30 carbon atoms, or an aromatic group such as an aryl or an alkylaryl group, at least one of the groups R₈ to R₁₁ denoting a group containing from 8 to 30 carbon atoms, preferably from 12 to 24 carbon atoms. The aliphatic groups can comprise heteroatoms such as, in particular, oxygen, nitrogen, sulphur and halogens. The aliphatic groups are, for example, selected from C₁-C₃₀ alkyl, C₁-C₃₀ alkoxy, polyoxy(C₂-C₆)alkylene, C₁-C₃₀ alkylamide, (C₁₂-C₂₂)alkylamido(C₂-C₆)alkyl, (C₁₂-C₂₂)alkyl acetate and C₁-C₃₀ hydroxyalkyl groups; X⁻ is an anion selected from the group of halides, phosphates, acetates, lactates, (C₁-C₄)alkyl sulphates and (C₁-C₄)alkyl or (C₁-C₄)alkylaryl sulphonates.

Among the quaternary ammonium salts of formula (XI), those that are preferred are, on the one hand, tetraalkylammonium salts, for instance dialkyldimethylammonium or alkyltrimethylammonium salts in which the alkyl group contains approximately from 12 to 22 carbon atoms, in particular behenyltrimethylammonium, distearyldimethyl-ammonium, cetyltrimethylammonium or benzyldimethylstearyl-ammonium salts, or, on the other hand, the palmitylamido-propyltrimethylammonium salt, the stearamidopropyltrimethyl-ammonium salt, the stearamidopropyldimethylcetearylammonium salt, or the stearamidopropyldimethyl(myristyl acetate)ammonium salt sold under the name Ceraphyl® 70 by the company Van Dyk. It is particularly preferred to use the chloride salts of these compounds;
quaternary ammonium salts of imidazoline, such as, for example, those of formula (XII) below:

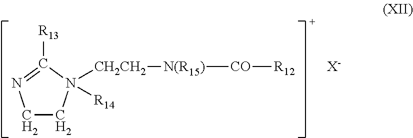

in which R₁₂ represents an alkenyl or alkyl group containing from 8 to 30 carbon atoms, for example tallow fatty acid derivatives, R₁₃ represents a hydrogen atom, a C₁-C₄ alkyl group or an alkenyl or alkyl group containing from 8 to 30 carbon atoms, R₁₄ represents a C₁-C₄ alkyl group, R₁₅ represents a hydrogen atom or a C₁-C₄ alkyl group, X⁻ is an anion selected from the group of halides, phosphates, acetates, lactates, alkyl sulphates, alkyl sulphonates or alkylaryl sulphonates, the alkyl and aryl groups of which preferably comprise, respectively, from 1 to 20 carbon atoms and from 6 to 30 carbon atoms. Preferably, R₁₂ and R₁₃ denote a mixture of alkenyl or alkyl groups containing from 12 to 21 carbon atoms, for example tallow fatty acid derivatives, R₁₄ denotes a methyl group and R₁₅ denotes a hydrogen atom. Such a product is, for example, sold under the name Rewoquat® W 75 by the company Rewo;
diquaternary or triquaternary ammonium salts of formula (XIII):

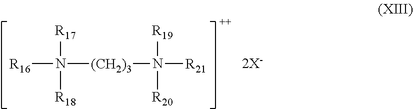

in which R₁₆ denotes an alkyl group containing from approximately 16 to 30 carbon atoms, which is optionally hydroxylated and/or interrupted with one or more oxygen atoms, R₁₇ is selected from hydrogen or an alkyl group containing from 1 to 4 carbon atoms or the following group:

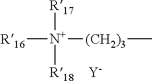

R′₁₆R′₁₇, R′₁₈, R₁₈, R₁₉, R₂₀ and R₂₁, which may be identical or different, are selected from hydrogen or an alkyl group containing from 1 to 4 carbon atoms, and X⁻ and Y⁻ are anions in particular selected from the group of halides, acetates, phosphates, nitrates and (C₁-C₆)alkyl sulphates, in particular methyl sulphate and ethyl sulphate. Such compounds are, for example, Finquat CT-P offered by the company Finetex (Quaternium-89), Finquat CT offered by the company Finetex (Quaternium 75) and Condicate CT offered by the company Innospec Active Chemicals (Quaternium-75);
quaternary ammonium salts containing at least one ester function, such as those of formula (XIV) below:

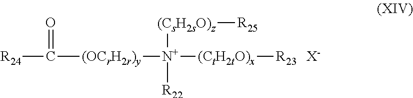

in which:

R₂₂ is selected from C₁-C₆ alkyl groups and C₁-C₆ hydroxyalkyl or dihydroxyalkyl groups;

R₂₃ is selected from

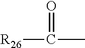

the group
the groups R₂₇ which are linear or branched, saturated or unsaturated C₁-C₂₂ hydrocarbon-based groups,
a hydrogen atom,

R₂₅ is selected from:
the group

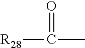

the groups R₂₉ which are linear or branched, saturated or unsaturated C₁-C₆ hydrocarbon-based groups,
a hydrogen atom,

R₂₄, R₂₆ and R₂₈, which may be identical or different, are selected from linear or branched, saturated or unsaturated C₇-C₂₁ hydrocarbon-based groups;

r, s and t, which may be identical or different, are integers from 2 to 6;

y is an integer from 1 to 10;

x and z, which may be identical or different, are integers from 0 to 10;

X⁻ is a simple or complex, organic or inorganic anion;

with the proviso that the sum x+y+z is from 1 to 15, that when x is 0, then Rₙ denotes R₂₇ and that when z is 0, then R₂₅ denotes R₂₉.

The R₂₂ alkyl groups may be linear or branched and more particularly be linear.

Preferably, R₂₂ denotes a methyl, ethyl, hydroxyethyl or dihydroxypropyl group, and more particularly a methyl or ethyl group.

Advantageously, the sum x+y+z is from 1 to 10.

When R₂₃ is a hydrocarbon-based group R₂₇, it may be long and contain from 12 to 22 carbon atoms, or short and contain from 1 to 3 carbon atoms.

When R₂₅ is a hydrocarbon-based group R₂₉, it preferably contains from 1 to 3 carbon atoms.

Advantageously, R₂₄, R₂₆ and R₂₈, which may be identical or different, are selected from linear or branched, saturated or unsaturated C₁₁-C₂₁ hydrocarbon-based groups, and more particularly from linear or branched, saturated or unsaturated C₁₁-C₂₁ alkyl and alkenyl groups.

Preferably, X and z, which may be identical or different, are 0 or 1.

Advantageously, y is equal to 1.

Preferably, r, s and t, which may be identical or different, are 2 or 3, and even more particularly are equal to 2.

The anion X⁻ is preferably a halide (chloride, bromide or iodide) or an alkyl sulphate, more particularly methyl sulphate. However, methanesulphonate, phosphate, nitrate, tosylate, an anion derived from an organic acid, such as acetate or lactate, or any other anion compatible with the ammonium containing an ester function, may be used.

The anion X⁻ is even more particularly chloride or methyl sulphate.

The ammonium salts more particularly used in the composition according to the invention are the ammonium salts of formula (XIV) in which:

R₂₂ denotes a methyl or ethyl group,

x and y are equal to 1;

z is equal to 0 or 1;

r, s and t are equal to 2;

R₂₃ is selected from:

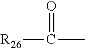

the group
methyl, ethyl or C₁₄-C₂₂ hydrocarbon-based groups,
a hydrogen atom;

R₂₅ is selected from:
the group

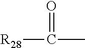

a hydrogen atom;

R₂₄, R₂₆ and R₂₈, which may be identical or different, are selected from linear or branched, saturated or unsaturated C₁₃-C₁₇ hydrocarbon-based groups, and preferably from linear or branched, saturated or unsaturated C₁₃-C₁₇ alkyl or alkenyl groups.

The hydrocarbon-based groups are advantageously linear.

Mention may be made, for example, of the compounds of formula (XIV) such as the diacyloxyethyldimethylammonium, diacyloxyethylhydroxyethylmethylammonium, monoacyloxyethyldi-hydroxyethylmethylammonium, triacyloxyethylmethylammonium and monoacyloxyethylhydroxyethyldimethylammonium salts (chloride or methyl sulphate in particular), and mixtures thereof. The acyl groups preferably contain 14 to 18 carbon atoms and are obtained more particularly from a plant oil such as palm oil or sunflower oil. When the compound contains several acyl groups, these groups may be identical or different.

These products are obtained, for example, by direct esterification of triethanolamine, triisopropanolamine, an alkyldi-ethanolamine or an alkyldiisopropanolamine, which are optionally oxyalkylenated, with C₁₀-C₃₀ fatty acids or with mixtures of C₁₀-C₃₀ fatty acids of plant or animal origin, or by transesterification of the methyl esters thereof. This esterification is followed by a quaternization using an alkylating agent such as an alkyl halide (preferably a methyl or ethyl halide), a dialkyl sulphate (preferably dimethyl or diethyl sulphate), methyl methanesulphonate, methyl para-toluenesulphonate, glycol chlorohydrin or glycerol chlorohydrin.

Such compounds are, for example, sold under the names Dehyquart® by the company Henkel, Stepanquat® by the company Stepan, Noxamium® by the company CECA, or Rewoquat® WE 18 by the company Rewo-Witco.

The composition according to the invention may contain, for example, a mixture of quaternary ammonium monoester, diester and triester salts, with a majority by weight of diester salts.

Mixtures of ammonium salts that can be used include, for example, the mixture containing 15% to 30% by weight of acyloxy-ethyldihydroxyethylmethylammonium methyl sulphate, 45% to 60% of diacyloxyethylhydroxyethylmethylammonium methyl sulphate and 15% to 30% of triacyloxyethylmethylammonium methyl sulphate, the acyl groups containing from 14 to 18 carbon atoms and being obtained from palm oil, which is optionally partially hydrogenated.

It is also possible to use the ammonium salts containing at least one ester function that are described in U.S. Pat. Nos. 4,874,554 and 4,137,180.

The particularly preferred cationic surfactant(s) that can be used according to the invention is (are) selected from the compounds of formula (XI) or of formula (XIV), methyl(C₉-C₁₉)alkyl(C₁₀-C₂₀)alkylamidoethylimidazolium salts and stearamidopropyldimethyl-amine.

Among all the cationic surfactants that may be present in the composition according to the invention, cetyltrimethylammonium, behenyltrimethylammonium, di(palmitoyloxyethyl)hydroxyethyl-methylammonium, di(stearoyloxyethyl)hydroxyethylmethylammonium and methyl(C₉-C₁₉)alkyl(C₁₀-C₂₀)alkylamidoethylimidazolium salts, the stearamidopropyltrimethylammonium salt, stearamidopropyl-dimethylamine and the stearamidopropyldimethylcetearylammonium salt, and mixtures thereof, are preferably selected.

Among the silicones that can be used as conditioning agents in accordance with the present invention, mention may be made, in a nonlimiting manner, of:

I. Volatile Silicones:

Said silicones have a boiling point of between 60° C. and 260° C. Among silicones of this type, mention may be made of:

(a) cyclic silicones containing from 3 to 7 silicon atoms, and preferably 4 to 5.

These are, for example, octamethylcyclotetrasiloxane sold under the name Volatile Silicone 7207® by the company Union Carbide or Silbione 70045 V2® by the company Rhone Poulenc, decamethyl-cyclopentasiloxane sold under the name Volatile Silicone 7158® by the company Union Carbide, Silbione 70045 V5® by the company Rhone Poulenc, and mixtures thereof. Mention is also made of cyclopolymers of the dimethylsiloxane/methylalkylsiloxane type, such as Volatile Silicone FZ 3109® sold by the company Union Carbide, which is a dimethylsiloxane/methyloctylsiloxane cyclo copolymer;

(b) linear volatile silicones containing 2 to 9 silicon atoms and having a viscosity of less than or equal to 5×10⁻⁶ m²/s at 25° C.

An example is the hexamethyldisiloxane sold under the name Silbione 70041 V0,65® by the company Rhone Poulenc. This type of product is described in the article by Todd & Byers “Volatile silicone fluids for cosmetics”, Cosmetics and Toiletries, Vol. 91, Jan 76, pages 27-32.

Ii. Non-Volatile Silicones

They are mainly constituted of polyalkylsiloxanes, polyaryl-siloxanes, polyalkylarylsiloxanes and organomodified polysiloxanes, and mixtures thereof. They may be in the form of oils, gums and resins.

Among the polyalkylsiloxanes, mention may mainly be made of linear polydimethylsiloxanes having a viscosity greater than 5×10⁻⁶ m²/s, and preferably less than 2.6 m²/s, i.e.:
containing trimethylsilyl end groups, for instance, and in a nonlimiting manner, the Silbione® oils of the 70047 series, sold by the company Rhone Poulenc, the Wacker Belsil DM 60 000 oil from Wacker, the Dow Corning 200 oils of viscosity 60 000 cSt or certain Viscasil® oils from the company General Electric,
containing trihydroxysilyl end groups, such as the oils of the 48 V® series from the company Rhone Poulenc.

In this class of polyalkylsiloxanes, mention may also be made of the polyalkylsiloxanes sold by the company Goldschmidt under the names Abilwax 9800® and Abilwax 9801®, which are poly(C₁-C₂₀)alkylsiloxanes.

Among the polyalkylarylsiloxanes, mention may be made of linear and/or branched polydimethylphenylsiloxanes and polydimethyldiphenylsiloxanes with a viscosity of 10⁻⁵ to 5×10⁻² m²/s, such as, for example:
the Rhodorsil® 763 oil from Rhone Poulenc,
the Silbione® oils of the 70641 series from Rhone Poulenc, such as the Silbione 70641 V30® and Silbione 70641 V200® oils,
the DC 556® Cosmetic Grade Fluid product from Dow Corning,
the silicones of the PK series from Bayer, such as PK20®,
the silicones of the PN and PH series from Bayer, such as PN 1000® and PH 1000®,
certain oils of the SF series from General Electric, such as SF 1250®, SF 1265®, SF 1154® and SF 1023®.

The silicone gums in accordance with the present invention are polydiorganosiloxanes having a high number-average molecular mass of between 200 000 and 1 000 000, used alone or as a mixture in a solvent selected from volatile silicones, polydimethylsiloxane (PDMS) oils, polyphenylmethylsiloxane (PPMS) oils, isoparaffins, methylene chloride, pentane, dodecane, tridecane and tetradecane, or mixtures thereof.

Mention is made, for example, of the compounds having the following structures:
poly[(dimethylsiloxane)/(methylvinylsiloxane)] gums,
poly[(dimethylsiloxane)/(diphenylsiloxane)] gums,
poly[(dihydrogenodimethylsiloxane)/(divinylsiloxane)] gums,
poly[(dimethylsiloxane)/(phenylmethylsiloxane)] gums,
poly[(dimethylsiloxane)/(diphenylsiloxane)/(methylvinyl-siloxane)] gums.

The Mirasil DM 300 000 gum from the company Rhodia may be mentioned.

Mention may also be made, for example, in a nonlimiting manner, of the following mixtures:

1) mixtures formed from a polydimethylsiloxane hydroxylated at the end of a chain (Dimethiconol according to the CTFA nomenclature), and from a cyclic polydimethylsiloxane (Cyclomethicone according to the CTFA nomenclature), such as the products Q2 1401® or Dow Corning 1501 Fluid, sold by the company Dow Corning;

2) mixtures formed from a polydimethylsiloxane gum with a cyclic silicone, such as the product SF 1214 Silicone Fluid® from General Electric, which is an SE 30® gum of MW 500 000 (-Mₙ) dissolved in SF 1202 Silicone Fluid® (decamethylcyclopentasiloxane);

3) mixtures of two PDMSs of different viscosities, in particular of a PDMS gum and of a PDMS oil, such as the products SF 1236® and CF 1241® from the company General Electric. The product SF 1236® is the mixture of an SE 30® gum defined above with a viscosity of 20 m²/s and of an SF 96® oil with a viscosity of 5×10⁶ m²/s (15% of SE 30® gum and 85% of SF 96® gum).

The product CF 1241® is the mixture of an SE 30® gum (33%) and of a PDMS (67%) with a viscosity of 10⁻³ m²/s.

The organopolysiloxane resins that can be used in accordance with the invention are crosslinked siloxane systems containing the units: R₂SiO_{2/2}, RSiO_{3/2} and SiO_{4/2} in which R represents a hydrocarbon-based group containing 1 to 6 carbon atoms or a phenyl group. Among these products, those which are particularly preferred are those in which R denotes a (C₁-C₄) lower alkyl group or a phenyl group.

Among these resins, mention may be made of the product sold under the name Dow Corning 593® or those sold under the names Silicone Fluid SS 4230 and Silicone Fluid SS 4267 by the company General Electric and which are dimethyl/trimethylpolysiloxanes.

The organomodified silicones in accordance with the present invention are silicones as defined above, comprising, in their general structure, one or more organofunctional groups directly attached to the siloxane chain or attached by means of a hydrocarbon-based group.

Mention is made, for example, of the silicones comprising:

a) perfluoro groups, such as trifluoroalkyls, for instance those sold by the company General Electric under the names FF.150 Fluorosilicone Fluid® or by the company Shin Etsu under the names X-22-819®, X-22-82®, X-22-821® and X-22-822®;

b) hydroxyacylamino groups, for instance those described in patent application EP 0 342 834, and in particular the silicone sold by the company Dow Corning under the name Q2-8413®;

c) thiol groups, such as the silicones X 2-8360® from the company Dow Corning or GP 72A® and GP 71® from Genesee;

d) substituted or unsubstituted non-quaternized amino groups, such as in GP 4 Silicone Fluid® from Genesee, GP 7100® from Genesee, Q2 8220® from Dow Corning, AFL 40® from Union Carbide or the silicone called Amodimethicone in the CTFA dictionary;

e) carboxylate groups, such as the products described in patent EP 186 507 from Chisso Corporation;

f) hydroxylated groups, such as the polyorganosiloxanes comprising a hydroxyalkyl function, described in patent application FR 85 16334, corresponding to formula (XV) below:

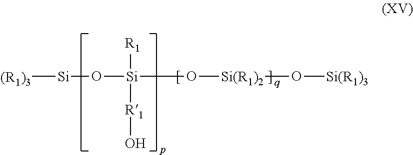

in which:
the R₁ groups, which may be identical or different, are selected from methyl and phenyl groups, at least 60 mol % of the R₁ groups being methyl;
the R′₁ group is a divalent hydrocarbon-based C₂-C₁₈ alkylene link;
p is between 1 and 30 inclusive;
q is between 1 and 150 inclusive.

Mention may most particularly be made of the product sold by Dow Corning under the name DC 190;

g) alkoxylated groups, such as in the silicone copolymer F 755® from SWS Silicones and the products Abilwax 2428®, Abilwax 2434® and Abilwax 2440® from the company Goldschmidt;

h) acyloxyalkyl groups, for instance the polyorganopoly-siloxanes described in patent application FR 88 17433, corresponding to formula (XVI) below:

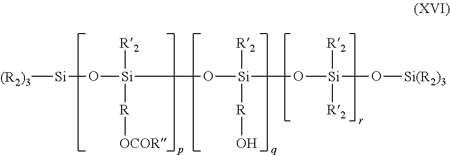

in which:
R₂ denotes methyl, phenyl, OCOR″ or hydroxyl, it being possible for only one of the R₂ per silicon atom to be OH;
R′₂ denotes methyl or phenyl, at least 60 mol % of the R₂ and R′₂ groups together being methyl;
R″ denotes C₈-C₂₀ alkyl or alkenyl;
R denotes a divalent, linear or branched C₂-C₁₈ hydrocarbon-based alkylene;
r is between 1 and 120 inclusive;
p is between 1 and 30 inclusive;
q is equal to 0 or is less than 0.5 p, p+q being between 1 and 30 inclusive;

the polyorganosiloxanes of formula (XVI) can comprise:

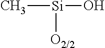

groups in proportions not exceeding 15% of the sum p+q+r;

i) quaternary ammonium groups, as in the products X2 81 08® and X2 81 09® and the product Abil K 3270® from the company Goldschmidt;

j) amphoteric or betaine groups, such as in the product sold by the company Goldschmidt under the name Abil B 9950®;

k) bisulphite groups, such as in the products sold by the company Goldschmidt under the names Abil S 201® and Abil S 255®;

l) polyethyleneoxy and/or polypropyleneoxy groups optionally comprising C₆-C₂₄ alkyl groups, such as the products called dimethicone copolyol, sold by the company Dow Corning under the name DC 1248, or the oils Silwet L 722, L 7500, L 77 and L 711 from the company Union Carbide, and the (C₁₂)alkyl methicone copolyol sold by the company Dow Corning under the name Q2 5200.

According to the invention, use may also be made of silicones comprising a polysiloxane portion and a portion consisting of a non-silicone organic chain, one of the two portions constituting the main chain of the polymer, the other being grafted onto said main chain. These polymers are, for example, described in patent applications EP-A-412 704, EP-A-412 707, EP-A-640 105, WO 95/00578, EP-A-582 152 and WO 93/23009 and patent applications U.S. Pat. No. 4,693,935, U.S. Pat. No. 4,728,571 and U.S. Pat. No. 4,972,037. These polymers are preferably anionic or non-ionic.

Such polymers are, for example, the copolymers that can be obtained by radical polymerization from the mixture of monomers constituted of:
a) 50% to 90% by weight of tert-butyl acrylate;
b) 0 to 40% by weight of acrylic acid;
c) 5% to 40% by weight of silicone macromer of formula (XVII):

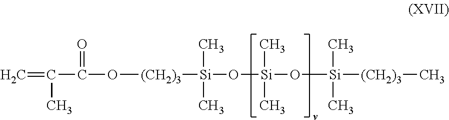

with v being a number ranging from 5 to 700; the percentages by weight being calculated relative to the total weight of the monomers.

Other examples of grafted silicone polymers are, in particular, polydimethylsiloxanes (PDMSs) onto which are grafted, by means of a connecting link of thiopropylene type, mixed polymer units of the poly((meth)acrylic acid) type and of the poly(alkyl(meth)acrylate) type; and polydimethylsiloxanes (PDMSs) onto which are grafted, by means of a connecting link of thiopropylene type, polymer units of the poly(isobutyl(meth)acrylate) type.

According to the invention, all the silicones can also be used in the form of emulsions, nanoemulsions or microemulsions.

The polyorganosiloxanes which are particularly preferred in accordance with the invention are:
non-volatile silicones selected from the family of polyalkylsiloxanes with trimethylsilyl end groups, such as oils having a viscosity of between 0.2 and 2.5 m²/s at 25° C., for instance the oils of the DC200 series from Dow Corning, in particular the one with a viscosity of 60 000 cSt, or of the Silbione 70047 and 47 series, and more particularly the oil 70 047 V 500 000, sold by the company Rhodia Chimie, and polyalkylsiloxanes with dimethylsilanol end groups, such as dimethiconols or polyalkylarylsiloxanes, for instance the oil Silbione 70641 V 200 sold by the company Rhodia Chimie;
polysiloxanes with amino groups, such as amodimethicones or trimethylsilyl amodimethicones.

The viscosities of the silicones can in particular be determined by the standard ASTM D445-97 (viscometry).

When the conditioning agent of the composition according to the invention is a hydrocarbon, said hydrocarbon is a linear or branched C₈-C₃₀ hydrocarbon.

Among the hydrocarbons which are liquid at ambient temperature and which correspond to this definition, mention may in particular be made of isododecane, isohexadecane and its isomers (such as 2,2,4,4,6,6-heptamethylnonane), isoeicosane, isotetracosane, the isomers of said compounds, n-nonadecane, n-dodecane, n-undecane, n-tridecane, n-pentadecane, and mixtures of these hydrocarbons.

Isododecane or an isomer thereof is preferably used according to the invention.

When the conditioning agent is a fatty alcohol, said fatty alcohol is of the linear or branched, saturated or unsaturated C₈-C₃₀ type. Among the latter, mention may, for example, be made of 2-butyloctanol, lauryl alcohol, 2-octyldodecanol, oleyl alcohol, isocetyl alcohol, isostearyl alcohol and behenyl alcohol, and mixtures thereof.

When the conditioning agent is a fatty ester, said fatty ester may be either an ester of a C₈-C₃₀ fatty acid and of a C₁-C₃₀ alcohol, and in particular an ester of a C₈-C₃₀ fatty acid and of a C₈-C₃₀ fatty alcohol, or an ester of a C₁-C₇ acid or diacid and of a C₈-C₃₀ fatty alcohol.

Among these esters, mention may, for example, be made of ethyl, isopropyl, 2-ethylhexyl and 2-octyldecyl palmitate, isopropyl, butyl, cetyl and 2-octyldecyl myristate, butyl and hexyl stearate, hexyl and 2-hexyldecyl laurate, isononyl isononanoate, dioctyl malate, myristyl myristate and cetyl palmitate, and mixtures thereof.

The ceramides or ceramide analogues, such as glycoceramides, that can be used as conditioning agent in the compositions according to the invention are known in themselves and are natural or synthetic molecules that can correspond to general formula (XVIII) below:

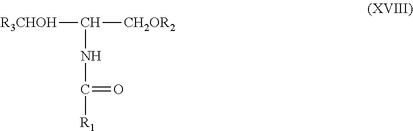

in which:
R₁ denotes a linear or branched, saturated or unsaturated alkyl group, derived from C₁₄-C₃₀ fatty acids, it being possible for this group to be substituted with a hydroxyl group in the alpha-position, or a hydroxyl group in the omega-position esterified with a saturated or unsaturated C₁₆-C₃₀ fatty acid;
R₂ denotes a hydrogen atom or a (glycosyl)ₙ, (galactosyl)ₘ or sulphogalactosyl group, in which n is an integer ranging from 1 to 4 and m is an integer ranging from 1 to 8;
R₃ denotes a C₁₅-C₂₆ hydrocarbon-based group, saturated or unsaturated in the alpha-position, it being possible for this group to be substituted with one or more C₁-C₁₄ alkyl groups;

it being understood that, in the case of natural ceramides or glycoceramides, R₃ can also denote a C₁₅-C₂₆ alpha-hydroxyalkyl group, the hydroxyl group being optionally esterified with a C₁₆-C₃₀ alpha-hydroxy acid.

The ceramides which are preferred in the context of the present invention are those described by Downing in Arch. Dermatol, Vol. 123, 1381-1384, 1987, or those described in French patent FR 2 673 179.

The ceramide(s) more particularly preferred according to the invention is (are) the compounds for which R₁ denotes a saturated or unsaturated alkyl derived from C₁₆-C₂₂ fatty acids; R₂ denotes a hydrogen atom; and R₃ denotes a linear, saturated C₁₅ group.

Such compounds are, for example:
N-linoleoyldihydrosphingosine,
N-oleoyldihydrosphingosine,
N-palmitoyldihydrosphingosine,
N-stearoyldihydrosphingosine,
N-behenoyldihydrosphingosine,

or mixtures of these compounds.

Even more preferably, ceramides for which R₁ denotes a saturated or unsaturated alkyl group derived from fatty acids; R₂ denotes a galactosyl or sulphogalactosyl group; and R₃ denotes a —CH═CH—(CH₂)₁₂—CH₃ group, are used.

By way of example, mention may be made of the product consisting of a mixture of these compounds, sold under the trade name Glycocer by the company Waitaki International Biosciences.

Among all these conditioning agents, use is preferably made of one or more conditioning agents selected from organosiloxanes and cationic polymers.

The cosmetic composition according to the invention preferably contains from 0.01% to 20% by weight, and more preferably from 0.05% to 10% by weight of conditioning agent(s) relative to the total weight of the cosmetic composition.

The cosmetic composition according to the invention may have a pH of between 3 and 10, and preferably between 5 and 7. This pH can be adjusted by means of acidifying agents and basifying agents, conventionally used in the cosmetics field.

The cosmetic composition used according to the invention may also contain one or more adjuvants that are customary in the cosmetics field, such as agents for preventing hair loss, anti-dandruff agents other than ellagic acid, ethers thereof and salts of these compounds, oxidizing agents, vitamins and provitamins, including panthenol, plant, animal, mineral or synthetic oils, waxes, sunscreens, coloured or uncoloured, mineral or organic pigments, dyes, pearlescent agents and opacifiers, sequestering agents, plasticizers, solubilising agents, antioxidants, hydroxy acids, fragrances, preservatives, and mixtures thereof.

The amounts of these various adjuvants are those conventionally used in the fields under consideration.

Of course, those skilled in the art will take care to select the optional compound(s) to be added to the composition according to the invention in such a way that the advantageous properties intrinsically associated with the composition in accordance with the invention are not, or not substantially, impaired by the addition envisaged.

Another subject of the invention consists of a cosmetic treatment method intended for eliminating and/or reducing dandruff, and in particular dandruff caused by yeasts of the Malassezia genus, characterized in that it comprises the application of a cosmetic composition comprising one or more compounds selected from ellagic acid, ethers thereof, ellagic acid salts and ellagic acid ether salts, and mixtures thereof, as an anti-dandruff agent, to the scalp. The cosmetic composition can then be optionally rinsed out with water. Preferably, this cosmetic treatment method is repeated at least twice a week.

The cosmetic composition is as previously defined.

The examples which follow are intended to illustrate the invention without, however, being limiting in nature.

### EXAMPLE 1

### Test of Effectiveness in an Aqueous-Alcoholic Gel by Leave-in Application

The aqueous-alcoholic gel for leave-in use A according to the invention was prepared according to the following table. The amounts are indicated as percent by weight of active material (A.M.) relative to the total weight of the composition.

| Leave-in composition A | % by weight of A.M. |
|---|---|
| Crosslinked carboxyvinyl | 0.3 |
| homopolymer (Carbopol Ultrez 10 | |
| from the company Lubrizol) | |
| 96 degrees ethyl alcohol | 17.4 |
| Ellagic acid | 0.5 |
| 2-amino-2-methyl-1-propanol | qs pH 7 |
| Water | qs 100 |

### Protocol:

The study was carried out on a group of 25 individuals who had scalps with dandruff displaying an excessive proliferation of yeasts of the Malassezia genus.

After two weeks of neutral shampoo, the individuals were treated with gel A of the invention or with an equal amount of a placebo comparative composition (identical composition without ellagic acid), at a rate of two applications per week.

The yeasts per unit surface area were counted using a corneodisc sample of squamae.

### Results:

After one week of treatment, it was observed that the use of ellagic acid formulated at 0.5% by weight in an aqueous-alcoholic gel resulted in a 56% reduction in the number of Malassezia yeasts present on the scalp, compared with a 29% reduction when the placebo composition without ellagic acid is used.

Consequently, the use of ellagic acid decreases, in a greater and more significant manner, the number of Malassezia yeasts present on the scalp after one week of treatment compared with the placebo comparative composition.

### EXAMPLE 2

### Test of Effectiveness in a Shampoo

The shampoo B according to the invention was prepared according to the following table. The amounts are indicated as percent by weight of active material (A.M.) relative to the total weight of the composition.

| Rinse-out composition B | % by weight of A.M. |
|---|---|
| Coconut acid | 0.24 |
| monoisopropanolamide (Empilan | |
| CIS sold by the company | |
| Huntsman) | |
| Cocamidopropylbetaine at 38% by | 2.4 |
| weight in an aqueous solution | |
| (Tego betain F50 sold by the | |
| company EvonikGoldschmidt) | |
| Sodium lauryl ether sulphate | 15.5 |
| comprising 2.2 mol of ethylene | |
| oxide at 26% by weight in an | |
| aqueous solution (Texapon AOS | |
| 225 UP sold by the company | |
| Cognis) | |
| Sodium chloride | 1.1 |
| Ellagic acid | 1 |
| Sodium benzoate | 0.5 |
| Salicylic acid | 0.2 |
| Polydimethylsiloxane 500 000 cSt | 2 |
| (molecular weight 250 000) | |
| (Silbione 70047V500000 sold by | |
| the company Bluestar) | |
| 1-(Hexadecyloxy)-2-octadecanol/ | 2.5 |
| cetyl alcohol mixture (Mexanyl | |
| GY sold by the company Chimex) | |
| Carboxyvinyl polymer (Carbopol | 0.3 |
| 980 sold by the company | |
| Lubrizol) | |
| Propylene glycol | 0.1 |
| Glycerol | 2 |
| pH agent | qs pH 5.3 |
| Fragrance | 0.5 |
| Water | qs 100 |

### Protocol:

The study was carried out on a group of 21 individuals who had scalps with dandruff displaying an excessive proliferation of yeasts of the Malassezia genus.

After two weeks of neutral shampoo, the shampoo composition B according to the invention was applied to the scalp for each individual. The composition was then rinsed out with water. This anti-dandruff treatment method was repeated twice a week for four weeks.

### Results:

Under the conditions of the study, the results obtained show that ellagic acid formulated at 1% by weight in a shampoo has a clear anti-dandruff effectiveness.

1. Use of one or more compounds selected from ellagic acid, ethers thereof, ellagic acid salts and ellagic acid ether salts, and mixtures thereof, as an anti-dandruff agent.

2. Use according to claim 1, for treating dandruff conditions associated with the proliferation of yeasts of the Malassezia genus on the scalp.

3. Use according to claim 1, characterized in that the salt(s) is (are) selected from alkali metal or alkaline-earth metal salts, in particular sodium, potassium, calcium or magnesium salts.

4. Use according to claim 1, characterized in that the ellagic acid ether(s) is (are) selected from mono-, di-, tri- or polyethers obtained by etherification of one or more hydroxyl groups of ellagic acid to give one or more OR groups, R being selected from C₂-C₂₀ alkyl groups, polyoxyalkylene groups, and groups derived from one or more monosaccharides or polysaccharides.

5. Use according to claim 1, characterized in that the anti-dandruff compound is ellagic acid or a salt thereof.

6. Use according to claim 1, characterized in that the compound(s) is (are) contained in a composition comprising a cosmetically acceptable medium consisting of water and optionally of cosmetically acceptable organic solvent(s).

7. Use according to claim 6, characterized in that the anti-dandruff compound(s) selected from ellagic acid, ethers thereof, ellagic acid salts and ellagic acid ether salts, represent(s) from 0.01% to 10%, preferably from 0.1% to 5%, and even better still from 0.2% to 2% by weight, relative to the total weight of the composition.

8. Use according to claim 6, characterized in that the composition comprises one or more thickeners.

9. Use according to claim 6, characterized in that the composition comprises one or more surfactants selected from anionic, non-ionic, amphoteric or zwitterionic surfactants, and mixtures thereof.

10. Use according to claim 6, characterized in that the composition is in the form of a gel and/or of a shampoo.

11. Use according to claim 6, characterized in that the composition also contains one or more conditioning agents selected from the group comprising cationic polymers, cationic surfactants, silicones such as organosiloxanes, linear or branched C₈-C₃₀ hydrocarbons, linear or branched C₈-C₃₀ fatty alcohols, esters of a C₈-C₃₀ fatty acid and of a C₁-C₃₀ alcohol, and in particular esters of a C₈-C₃₀ fatty acid and of a C₈-C₃₀ alcohol, esters of a C₁-C₇ acid or diacid and of a C₈-C₃₀ fatty alcohol, ceramides or ceramide analogues, and mixtures of these compounds.

12. Use according to claim 6, characterized in that the composition also contains at least one adjuvant selected from agents for preventing hair loss, anti-dandruff agents other than the ellagic acid, ethers thereof, ellagic acid salts and ellagic acid ether salts, oxidizing agents, vitamins and provitamins, including panthenol, plant, animal, mineral or synthetic oils, waxes, sunscreens, coloured or uncoloured, mineral or organic pigments, dyes, pearlescent agents and opacifiers, sequestering agents, plasticizers, solubilising agents, antioxidants, hydroxy acids, fragrances, preservatives, acidifying agents, and basifying agents, and mixtures thereof.

13. Cosmetic treatment method intended for eliminating and/or reducing dandruff, and in particular dandruff caused by yeasts of the Malassezia genus, characterized in that it comprises the application of a composition comprising from 0.01% to 10% by weight, relative to the total weight of the composition, of at least one compound selected from ellagic acid, ethers thereof, salts of these compounds and mixtures thereof, as defined in claim 6, to the scalp, optionally followed by rinsing out with water.
